(19) United States
(12) Patent Application Publication
(10) Pub. No.: US 2011/0120476 A1
(43) Pub. Date: May 26, 2011

(54) SLEEP APPLIANCE

(76) Inventor: Bryan Keropian, Tarzana, CA (US)

(21) Appl. No.: 12/996,745

(22) PCT Filed: Jun. 24, 2009
(86) PCT No.: PCT/US09/48495

§ 371 Date: Dec. 7, 2010

Related U.S. Application Data
(60) Provisional application No. 61/075,508, filed on Jun. 25, 2008.

(51) Int. Cl. A61F 5/56

(52) U.S. Cl. 128/848

(57) ABSTRACT

A dental oral appliance for patients who suffer with sleep disorders, to reduce or eliminate snoring or obstructive sleep apnea and keep the airways open during sleep Retention for the appliance is provided by an occlusal coverge of the upper or lower teeth A raised incisor ramp that extends from the incisal tip (biting edge) of the incisors toward the lingual, or posterior ramps, separate the upper and lower teeth to open the airway A transpalatal bar, which extends from the inside (lingual) of the right molars to the inside of the left molars, inhibits the upward/backward movement of the tongue. A longitudinal tongue restrainer is attached to the raised incisor ramp or the front of the appliance at the anterior end, and extends above, below or through the transpalatal bar near the posterior end, and aids in inhibiting and restraining the upward/backward movement of the tongue

This application claims the benefit of U.S. Provisional Application No. 61/075,508, filed Jun. 25, 2008, which application is incorporated herein by reference in its entirety.

### TECHNICAL FIELD

This invention relates to an intra-oral device for reducing or eliminating snoring and/or sleep apnea.

### BACKGROUND ART

The invention described in this application is an improvement over the devices described in my U.S. Pat. No. 6,766,802, issued Jul. 27, 2004, my U.S. Pat. No. 7,451,767, issued Nov. 18, 2008, my pending U.S. patent application Ser. No. 12/300,379, filed Nov. 11, 2008, and my pending U.S. patent application Ser. No. 12/102,239, filed Apr. 14, 2008.

As stated in my patents and applications referenced above, it has been estimated that ninety million American adults and children snore and that three in every ten adults snores. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death.

Obstructive sleep apnea occurs during sleep when the tongue falls and rolls upward and backward, blocking the airway for 10-90 seconds. These events are measured by spending the night sleeping in a center which measures the number of air blockage events per hour. Less than 5 events per hour is normal. 5-19 events per hour is mild sleep apnea. 20-39 events per hour is moderate sleep apnea. Over 40 events per hour is severe sleep apnea. For sleep apnea there are three main treatments of choice: the CPAP machine, surgery and oral sleep appliances. They are all designed to open the airway during sleep so that there is minimal or no air blockage.

Snoring is caused by vibration of the tissues due to air turbulence as the airway narrows and may be a sign that a patient is suffering from apnea. But not all snorers suffer from apnea. Snoring can be categorized by its severity. There is the snorer who snores but experiences no physical problems. Then, there is the snorer who suffers from apnea, or the snorer who suffers from upper airway resistance. In some of these people, though they may not actually experience apneic episodes, their snoring is so loud and their breathing so labored, that it still wakes them, and their partners, numerous times throughout the night.

Many spouses, partners and/or children suffer through the night from the annoying noise of the snorer. Snoring not only disturbs the sleeping pattern of the snorer himself, it is also disruptive to the family life by causing lack of sleep to all involved. This leaves all involved unrefreshed, tired and sleepy throughout the day. It can cause sleepiness while driving, reading, working or doing other tasks.

A broad variety of intra-oral and dental appliances and devices are now available to treat a patient for snoring. Some known oral devices for treating snoring and obstructive sleep apnea are worn inside of the mouth and work by repositioning of the jaw, moving the mandible, lifting the soft palate or moving the tongue forward. The various classes of treatment devices that now exist include mandibular advancers and tongue advancers. These appliances work by advancing the tongue and soft palate away from the back wall of the throat. Other methods used to treat snoring include controlled positive air-flow pressure systems, also known as CPAP, which require a nose mask and which are quite uncomfortable.

Other treatments for snoring include various surgeries, which are drastic steps to take to attempt to cure the problem, however snoring can be so disruptive to a person's life and relationships, that some sufferers resort to surgery.

### BRIEF DESCRIPTION OF THE INVENTION

The sleep appliance of this invention is a dental oral appliance for use with patients who suffer with sleep disorders. Primarily it is designed to reduce or eliminate snoring and to open the airway for a sleeping individual who suffers with obstructive sleep apnea. Embodiments of the appliance are physically designed similar to an upper (maxillary) or lower (mandibular) bruxism appliance. They cover the inside (lingual) of the upper teeth and have an open palate (nothing covering the middle area of the palate) or the inside (lingual) of the lower teeth. The body of the appliance has a series of recesses to fit against the lingual side of the teeth.

Retention (holding ability) for the appliance is provided by acrylic fittings which hold the appliance in place in the same manner as an occlusal night guard.

In one embodiment, in the anterior area there is a raised strip or ramp that extends from the incisal tip (biting edge) of two or more of the incisors toward the lingual. It extends back a short distance from the incisors (where they meet or touch each other). This raised anterior strip acts as a bite discluder, to disclude or separate the posterior teeth.

In an additional embodiment there is no anterior ramp. The upper and lower teeth are separated by raised posterior ramps. This embodiment allows more room for the tongue to come forward, if desired.

There is a transverse strip, a transpalatal bar, that extends from the inside (lingual) of the upper or lower right molars to the inside of the upper or lower left molars. This transverse strip extends from the right to the left and covers the tongue. It does not touch the tongue unless the tongue attempts to move upward or backward, as often happens during sleep, causing snoring or sleep apnea. The transpalatal bar inhibits and restrains the upward and backward movement of the tongue, keeping the airway open during sleep.

Optionally, the transpalatal bar is slightly curved upward at its center, or it can be straight across, so that it does not touch the tongue but passes just over the tongue when the tongue is in its normal position. Also, the transpalatal bar does not touch the palate. By not touching either the tongue or the palate, the device of this invention is comfortable to wear and easily tolerated by patient users. Prior art devices, which have pushed the palate up, were found to be unusable, as they often were so uncomfortable that they were unable to be tolerated by the user. The exact radius of the curvature of the transpalatal bar is determined by the physical dimensions and structure of each individual patient's anatomy. Some patients may need little or no curvature to achieve optimum results and other patients may need more curvature. The object is to have the transpalatal bar not touch the tongue or the palate, so that it will be tolerated, but inhibit and restrain any upward or backward movement of the tongue during sleep.

In addition, in certain devices of this invention, there is optionally a posterior tongue restrainer (a tail) that extends backward from the center of the transpalatal bar. This posterior tongue restrainer provides a further barrier to the tongue's superior and posterior movement that blocks the airway to the posterior portion of the mouth. The posterior tongue restrainer, like the transpalatal bar, does not touch the tongue in its normal position but does restrain and inhibit the upward and backward movement of the tongue during sleep. Also, the posterior tongue restrainer, like the transpalatal bar, does not touch the palate. This posterior tongue restrainer may be added to all of the appliances that are described in my issued patent and in my co-pending applications set forth above. The need for a posterior tongue restrainer depends on the needs of the patient. There may be one posterior tongue restrainer or a plurality of posterior tongue restrainers extending back from the transpalatal bar.

Alternatively, the transpalatal bar may be removed and reattached to another position, using common dental acrylic. With this adjustment, the appliance can be customized to each patient, to provide the proper fit for inhibiting upward and backward movement of the tongue of each patient at its maximum effectiveness.

The above described sleep appliances may also comprise an anterior tongue restrainer. This restrainer is connected to the transpalatal bar at the rear of the appliance, preferably the middle of the transpalatal bar, and to either the incisor raised strip or ramp discussed above or to the anterior portion of the appliance, generally lingual of teeth 8 and 9. The anterior tongue restrainer may be straight or curved slightly downward. It does not touch the tongue in its normal position nor does it touch the palate but it does inhibit and restrain the upward and backward movement of the tongue. It is utilized for those patients who need extra assistance in blocking the upward and backward movement of the tongue during sleep, and it adds to the clinical success in relieving snoring and Obstructive Sleep Apnea problems.

Thus, there is a gap between each of the transpalatal bar, the posterior tongue restrainer and the anterior tongue restrainer, and the palate and also a gap between the transpalatal bar, the posterior tongue restrainer and the anterior tongue restrainer, and the tongue in its normal position.

An additional embodiment described in my prior applications, comprises one or both of two additional elements. The first element is a transverse (horizontal) tail piece attached to the rear end of an existing tail. The transverse tail piece is at right angles (perpendicular) to a tail which extends back from the transpalatal bar. The transverse tail piece aids in impeding the upward and back ward movement of the tongue during sleep. It also does not touch the tongue in its normal position.

The second improvement comprises a tail piece that extends vertically downward from the end of an existing tail. It has been discovered that most patients who are treated for snoring and sleep apnea respond favorably, to at least some extent, with at least one of the appliances described above. Some patients do not respond effectively to any of the previous designs. They still have serious snoring and sleep apnea problems. This vertically extending tail is one of the appliances that is designed to press against the tongue to push it down and hold the airway open.

Applicant has discovered in treating hundreds of patients that those patients who have the most serious snoring and sleep apnea problems, who are not sufficiently aided by any of the previously described appliances, are most able to tolerate the vertical tail piece pressing down on the rear portion of their tongue.

The new embodiments described herein are improved appliances that have a longitudinal tongue restrainer and fit on the lower teeth, as opposed to the upper teeth. The appliances have a transpalatal bar that crosses over the tongue from right to left. The transpalatal bar may be straight or may arch upward over the tongue toward the palate, depending upon the structure of the patient's oral cavity. However, the transpalatal bar, whether it is straight or arches over the tongue does not touch the palate, nor does it touch the tongue in most cases, however it does inhibit and restrain the upward and backward movement of the tongue during sleep.

A posterior tongue restrainer may be added to this straight or arched transpalatal bar.

To understand the effectiveness of the appliance, the mechanism of snoring and obstructive sleep apnea must be understood. While we sleep, the tongue falls back and up towards the palate and it partially or completely obstructs or closes the airway path. This results in snoring, obstructive sleep apnea, or Upper Airway Resistance Syndrome. The medical treatment for these maladies range from medication to a CPAP (Continuous Positive Airway Pressure) machine. The CPAP is nearly 100% successful when utilized. Unfortunately, the non-compliance for CPAP use ranges from 50% to 80% depending where one searches in the literature. The American Association of Sleep Medicine designated dental sleep appliances as the number one alternative to CPAP for mild and moderate sleep apnea.

The sleep appliance of this invention is designed to treat the problem of tongue blockage when sleeping. It works by utilizing several factors. First, it changes the vertical dimension (height of the opening or separation of the teeth). This results in an increased opening of the airway. Second, the transpalatal bar, the posterior tongue restrainer, the longitudinal tongue restrainer and the vertical tail piece, when needed, all act to effectively inhibit and restrain the upward and backward movement of the tongue, which would block the airway opening during sleep. Optionally, if needed, the sleep appliance can also include mandibular advancement to increase the opening, thus increasing the opening of the airway.

### OBJECTS OF THE INVENTION

Accordingly, several objects and advantages of the invention are as follows:

It is an object of this invention to provide a simple device to prevent or reduce snoring as well as Obstructive Sleep Apnea.

It is another object of this invention to provide a device, easily applied and easily tolerated, which will substantially prevent snoring.

Further objects and advantages will become apparent from a consideration of the following description and drawings.

### BRIEF DESCRIPTION OF THE DRAWINGS

FIG. 1 is a perspective view of the top of an embodiment of the improved sleep appliance of this invention;

FIG. 2 is a rear view of the embodiment of FIG. 1;

FIG. 3 is a side elevation view of the embodiment of FIG. 1;

FIG. 4 is a perspective view of the top of another embodiment of the invention;

FIG. 5 is a rear view of the embodiment of FIG. 4;

FIG. 6 is a side elevation view of the embodiment of FIG. 4;

FIG. 7 is a perspective view of the top of another embodiment of the invention;

FIG. 8 is a rear view of the embodiment of FIG. 7;

FIG. 9 is a side elevation view of the embodiment of FIG. 7;

FIG. 10 is a perspective view of the top of another embodiment of the invention;

FIG. 11 is a rear view of the embodiment of FIG. 10;

FIG. 12 is a side elevation view of the embodiment of FIG. 10;

FIG. 13 is a perspective view of the top of another embodiment of the invention;

FIG. 14 is a rear view of the embodiment of FIG. 13;

FIG. 15 is a side elevation view of the embodiment of FIG. 13;

FIG. 16 is a perspective view of the top of another embodiment of the invention;

FIG. 17 is a rear view of the embodiment of FIG. 16;

FIG. 18 is a side elevation view of the embodiment of FIG. 16;

FIG. 19 is a perspective view of the top of another embodiment of the invention;

FIG. 20 is a rear view of the embodiment of FIG. 19;

FIG. 21 is a side elevation view of the embodiment of FIG. 19;

FIG. 22 is a perspective view of the top of another embodiment of the invention;

FIG. 23 is a rear view of the embodiment of FIG. 22;

FIG. 24 is a side elevation view of the embodiment of FIG. 22;

FIG. 25 is a perspective view of the top of another embodiment of the invention;

FIG. 26 is a rear view of the embodiment of FIG. 25;

FIG. 27 is a side elevation view of the embodiment of FIG. 25;

FIG. 28 is a perspective view of the top of another embodiment of the invention;

FIG. 29 is a rear view of the embodiment of FIG. 28;

FIG. 30 is a side elevation view of the embodiment of FIG. 28;

FIG. 31 is a perspective view of the top of another embodiment of the invention;

FIG. 32 is a rear view of the embodiment of FIG. 31;

FIG. 33 is a side elevation view of the embodiment of FIG. 31;

FIG. 34 is a perspective view of the top of another embodiment of the invention;

FIG. 35 is a rear view of the embodiment of FIG. 34;

FIG. 36 is a side elevation view of the embodiment of FIG. 34;

FIG. 37 is a perspective view of the top of another embodiment of the invention;

FIG. 38 is a rear view of the embodiment of FIG. 37;

FIG. 39 is a side elevation view of the embodiment of FIG. 37;

FIG. 40 is a perspective view of the top of another embodiment of the invention;

FIG. 41 is a rear view of the embodiment of FIG. 40;

FIG. 42 is a side elevation view of the embodiment of FIG. 40;

FIG. 43 is a perspective view of the top of another embodiment of the invention;

FIG. 44 is a rear view of the embodiment of FIG. 43;

FIG. 45 is a side elevation view of the embodiment of FIG. 43;

FIG. 46 is a perspective view of the top of another embodiment of the invention;

FIG. 47 is a rear view of the embodiment of FIG. 46;

FIG. 48 is a side elevation view of the embodiment of FIG. 46;

FIG. 49 is a perspective view of the top of another embodiment of the invention;

FIG. 50 is a rear view of the embodiment of FIG. 49;

FIG. 51 is a side elevation view of the embodiment of FIG. 49;

FIG. 52 is a perspective view of the top of another embodiment of the invention;

FIG. 53 is a rear view of the embodiment of FIG. 52;

FIG. 54 is a side elevation view of the embodiment of FIG. 52;

FIG. 55 is a side elevation view of another embodiment of the invention; and,

FIG. 56 is a side elevation view of another embodiment of the invention.

### BEST MODE FOR CARRYING OUT THE INVENTION

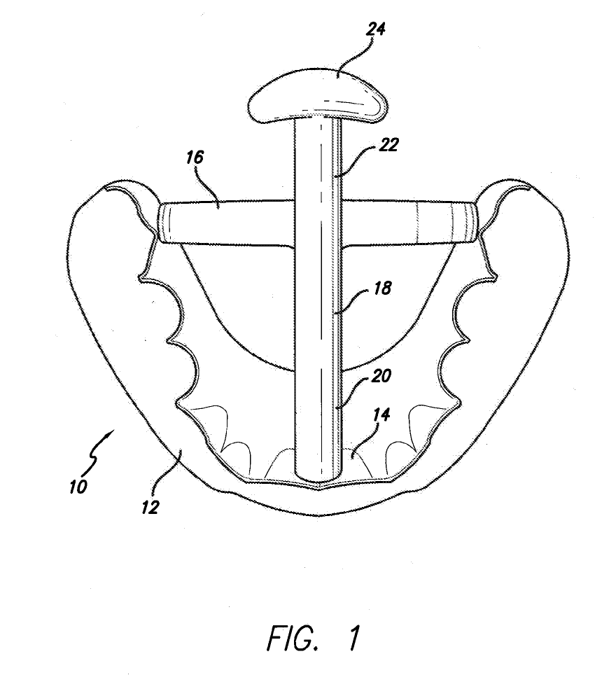

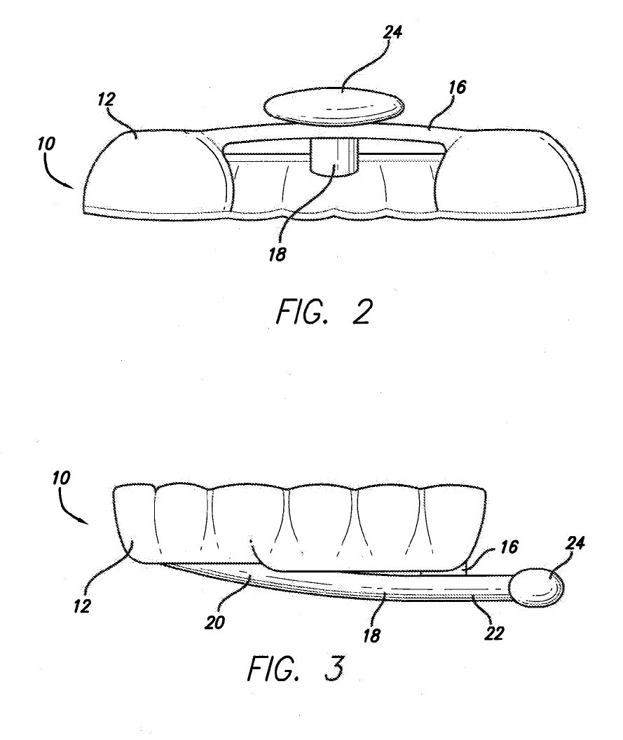

Referring now to FIGS. 1-3, there is shown a first embodiment 10 of the sleep appliance of this invention comprising a body 12. Body 12 is made entirely of an acrylic plastic, commonly used for dental devices, and is custom fitted to fit over the wearer's lower posterior teeth in the same manner as an occlusal night guard, which uses an occlusal coverage. The occlusal coverage holds appliance 10 firmly onto the lower posterior teeth.

There is a raised anterior strip 14 that extends from the incisal tip (biting edge) of two or more of the incisors toward the lingual. Strip 14 extends back a short distance from the middle of the central incisors. Strip 14 acts as a bite discluder, separating the posterior teeth. Strip 14 is preferably from about 3 mm to about 5 mm thick in order to separate the posterior teeth.

Transverse transpalatal bar 16 extends from the inside of the right lower molars to the inside of the left lower molars and inhibits the upward and backward movement of the tongue, to keep the airway open during sleep. Transpalatal bar 16 may be straight or curved upwards over the tongue, depending upon the needs of the patient. Transpalatal bar 16 does not touch the tongue in most cases and does not touch the palate. Transpalatal bar 16 does inhibit and restrain the upward and backward movement of the tongue.

Longitudinal tongue restrainer 18 extends and runs from the front (anterior) end of appliance 10 rearward, past transverse transpalatal bar 16. Longitudinal tongue restrainer 18 is a combination anterior tongue restrainer, the front (anterior) portion 20 forward of transpalatal bar 16, and the rear (posterior) portion 22, rearward of transpalatal bar 16. The front (anterior) portion 20 of longitudinal tongue restrainer 18 is attached to either raised anterior strip 14, as shown, or to the front of appliance 10. Longitudinal tongue restrainer 18, near its posterior end 22, is attached to the center area of transpalatal bar 16. In this embodiment, it is attached to the top of transpalatal bar 16. Longitudinal tongue restrainer 18 may be straight or curved downward towards the tongue, depending upon the needs of the patient, to further inhibit the upward and backward movement of the tongue. In this embodiment it is slightly curved downward towards the tongue, as shown best in FIG. 3. In addition, appliance 10 may be made with or without longitudinal tongue restrainer 18, depending upon the needs of the patient. In addition, longitudinal tongue restrainer 18 can touch the tongue in some cases.

Attached to the posterior section 22 of longitudinal tongue restrainer 18 is a perpendicular cross bar 24 attached at the rear end thereof. Posterior section 22 and cross bar 24 do not touch the tongue in most cases and do not touch the palate. They do inhibit the upward and backward movement of the tongue during sleep. For certain patients cross bar 24 is necessary to aid in inhibiting the upward and backward movement of the tongue during sleep. In this embodiment perpendicular cross bar 24 has a curved perimeter shape, called a “whale's 275 tail”. In addition, cross bar 24 can touch the tongue in some cases.

Longitudinal tongue restrainer 18 is attached to strip 14 and transpalatal bar 16 by the use of any convenient adhesive such as an acrylic.

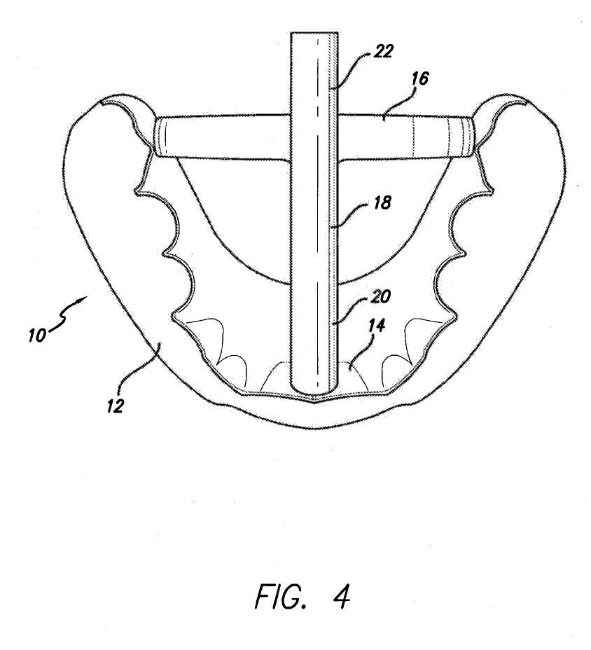

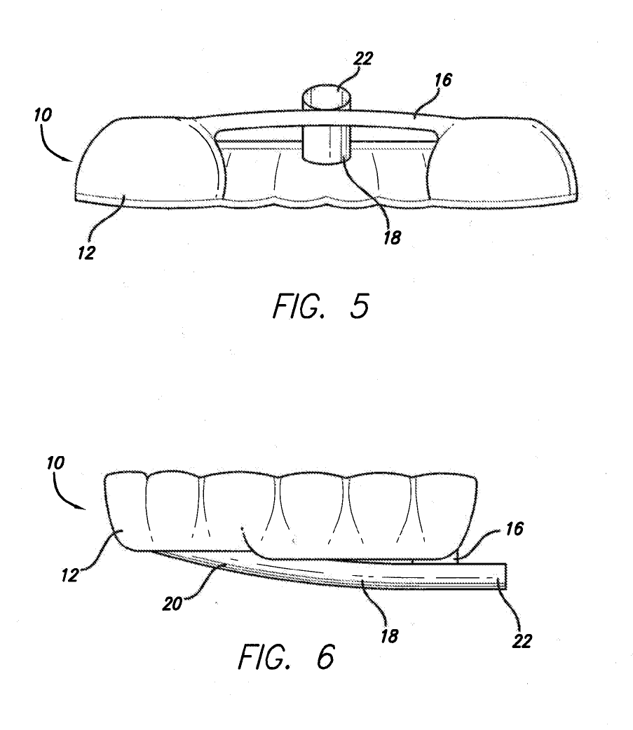

Referring to FIGS. 4-6, there is shown another embodiment of the appliance 10 which is the same as the embodiment of FIGS. 1-3, except that cross bar 24 is missing. If a patient does not need cross bar 24 for effective treatment, it may be excluded.

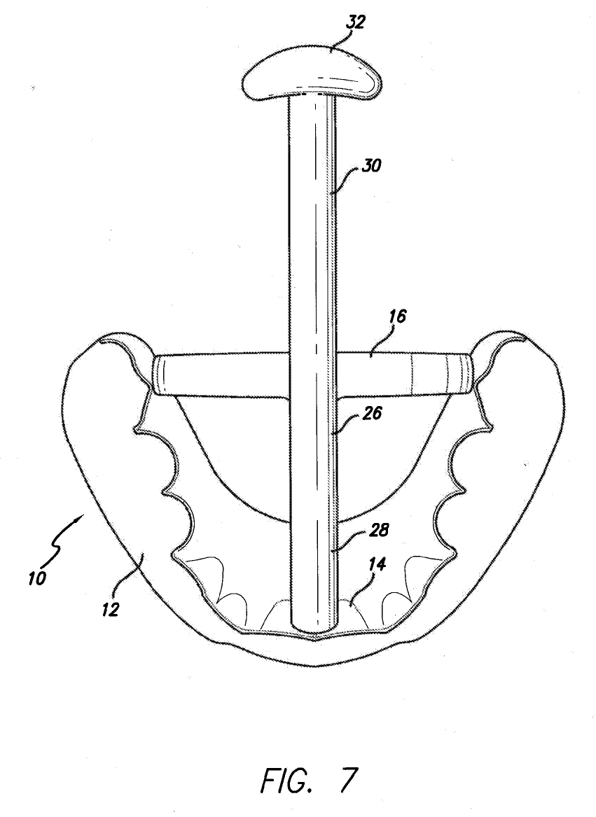

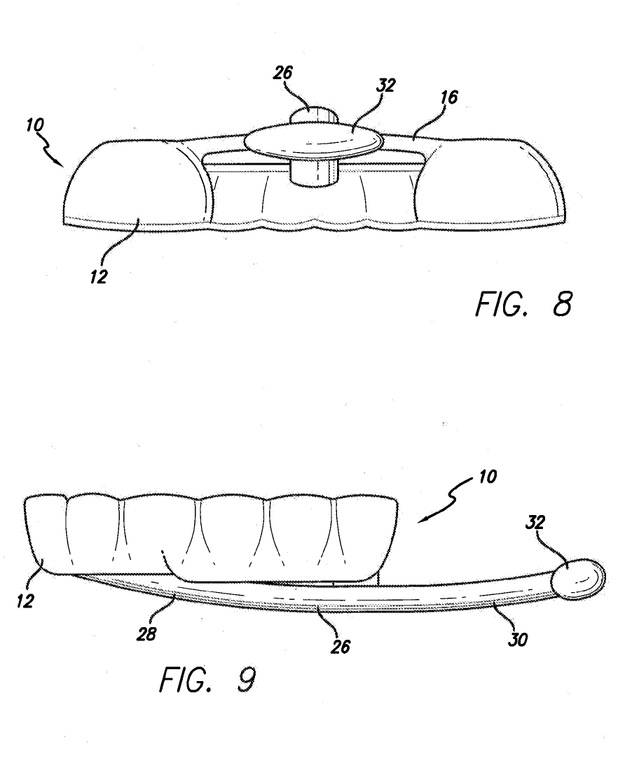

Referring now to FIGS. 7-9 there is shown another embodiment of the appliance 10, which is basically the same as the embodiment of FIGS. 1-3, except that longitudinal tongue restrainer 26 has a similar anterior portion 28 but an elongated posterior portion 30. Posterior portion 30 has a whale's tail 32.

The length of the posterior tongue restrainer portion 22 or 30, which can extend from about ¼ inch to about 2 ½ inches in length from the rear of transpalatal bar 16, and be about ¼inch to about 1 inch wide, from side to side, and the presence or absence of the cross-bar 24 or 32, which can be from about ¼ inch to about 2 inches in length, depends upon the needs of the patient. Whether the patient needs a short or an elongated posterior tongue restrainer portion must be determined by the medical practitioner trying different models on the patient until it is determined just what model works best in preventing snoring, while at the same time, being comfortable and easily tolerated by the patient. The posterior tongue restrainer 22 or 30 and the cross bar 24 or 32, may, or may not, touch the tongue, or may press on the tongue in either one spot or the entire length, depending upon the needs of the patient.

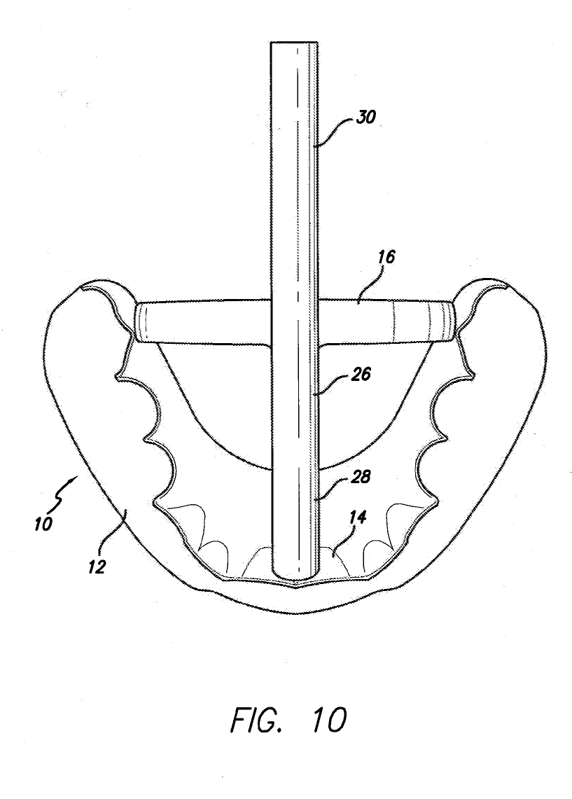

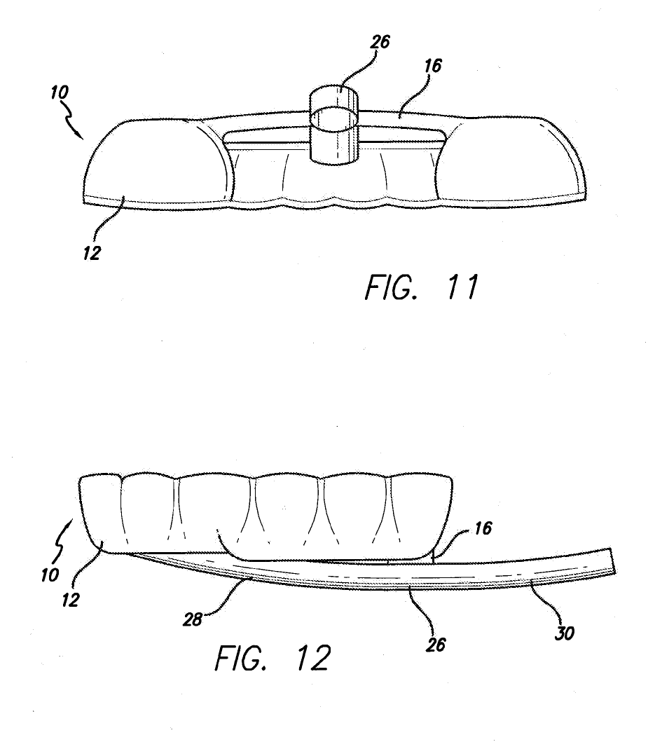

Referring now to FIGS. 10-12, there is shown an embodiment which is the same as FIGS. 7-9, except that the posterior cross-bar 32 is not utilized.

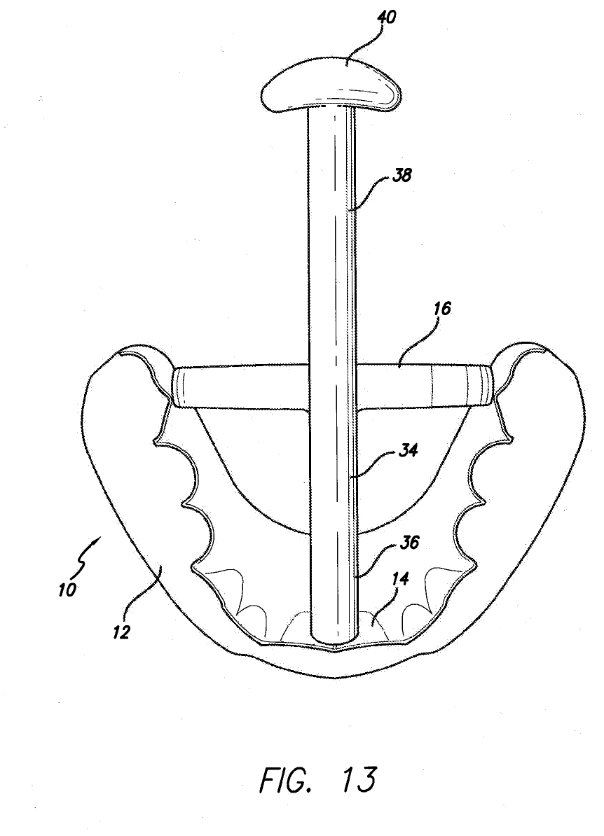

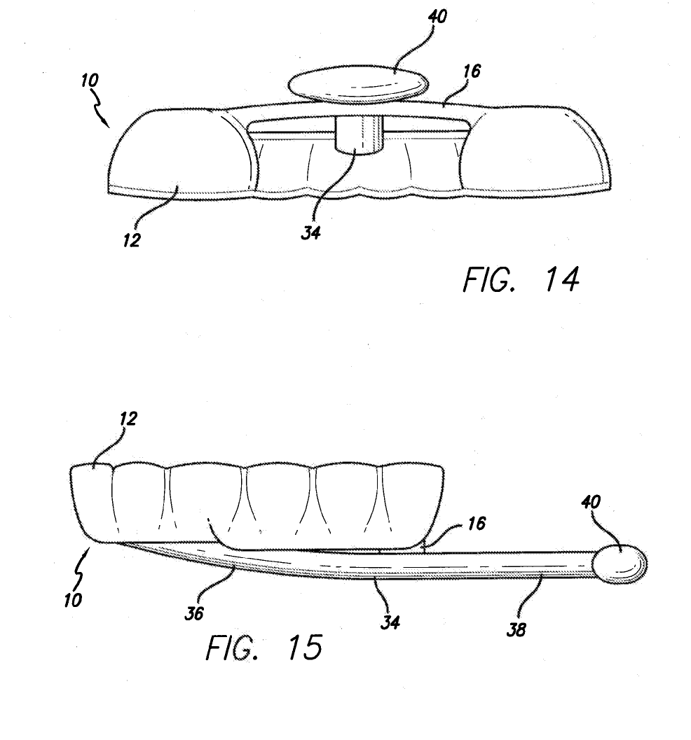

Referring now to FIGS. 13-15, there is shown an embodiment, similar to FIG. 7, in which longitudinal tongue restrainer 34 is elongated but the posterior portion 38 is straight, as compared to curved as in FIG. 7. Anterior portion 36 remains slightly curved as in FIGS. 7 and whale's tail 40 is present. The purpose of this embodiment is simply one that may work better for the patient than the embodiment of FIG. 7.

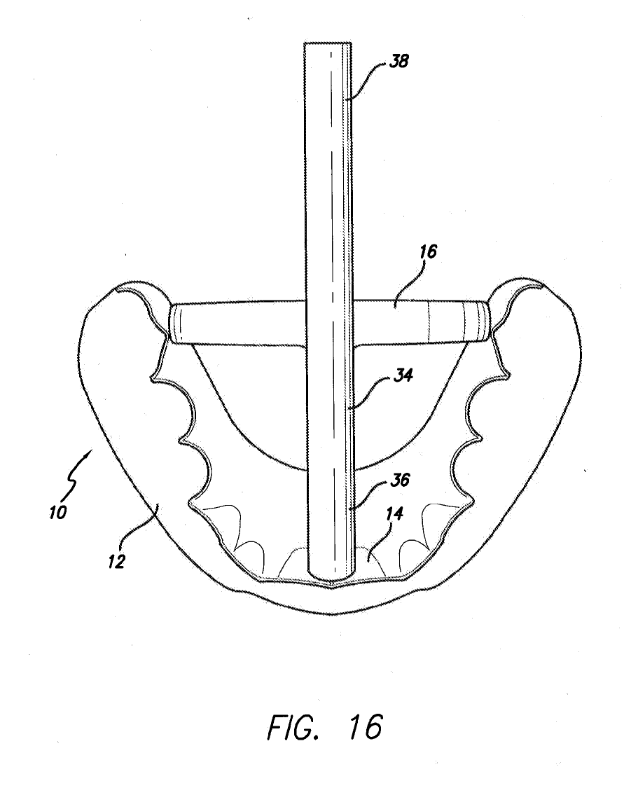

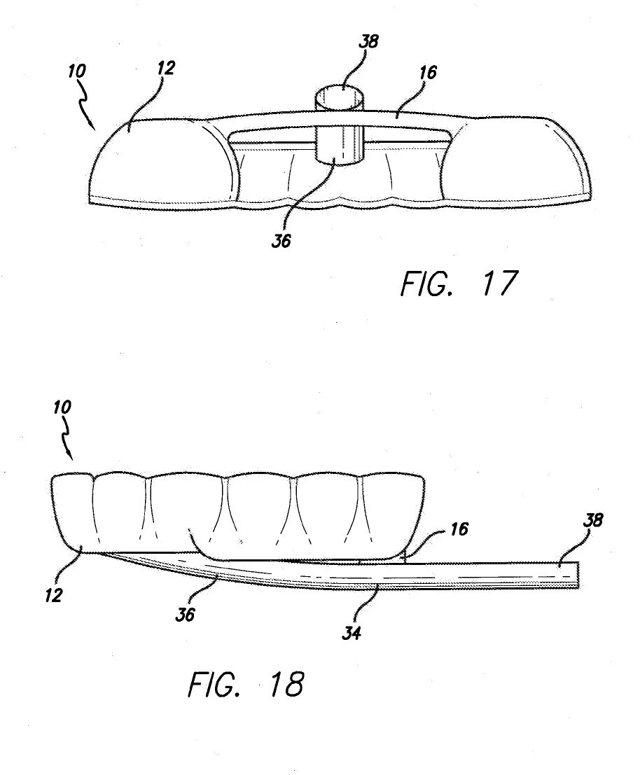

Referring now to FIGS. 16-18, there is shown an embodiment which is the same as FIG. 13, but the whale's tail 40 is not utilized,

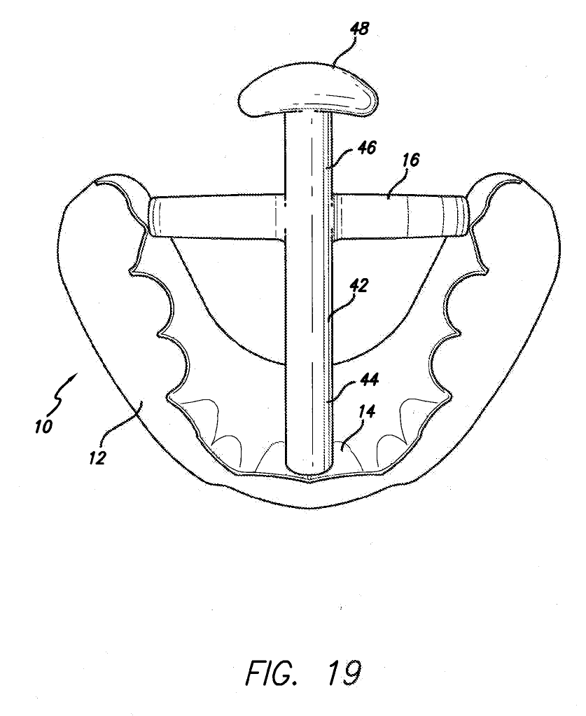

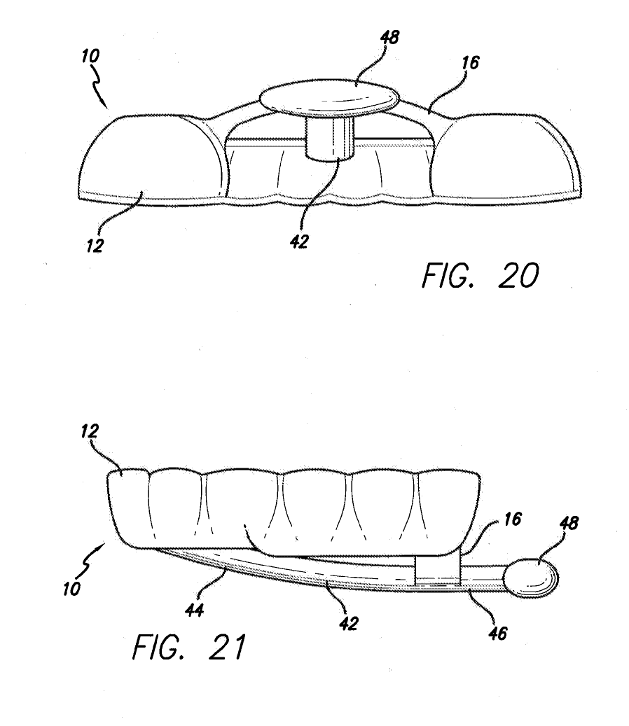

Referring now to FIGS. 19-21, there is shown an appliance 10, with raised anterior strip 17 and transpalatal bar 16. Longitudinal tongue restrainer 42 has anterior portion 44, which is attached to anterior strip 14, and posterior portion 46. Rather than sitting on the top of transpalatal bar 16, as in the prior described embodiments, posterior portion 46 extends through trnaspalatal bar 16. This is accomplished by longitudinal tongue restrainer 42 being formed together with transpalatal bar 16, as a single piece, usually from an acrylic. A whale's tail 48 is attached to the end of posterior portion 46. In addition, longitudinal tongue restrainer 42 and cross bar 48 may touch the tongue in some cases.

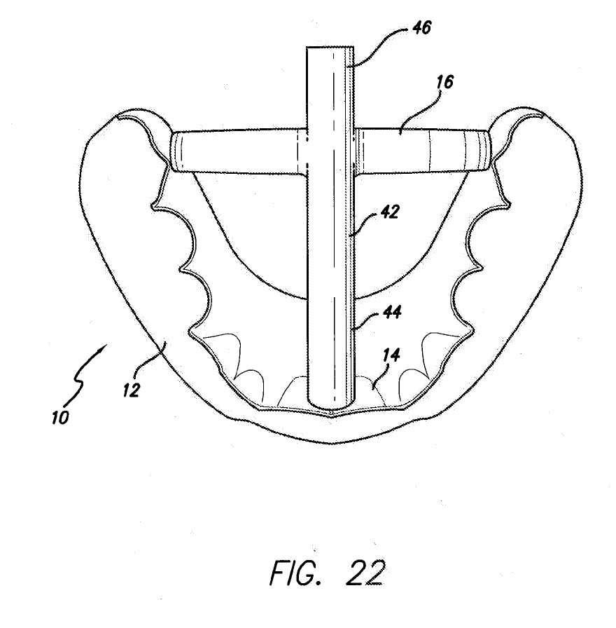

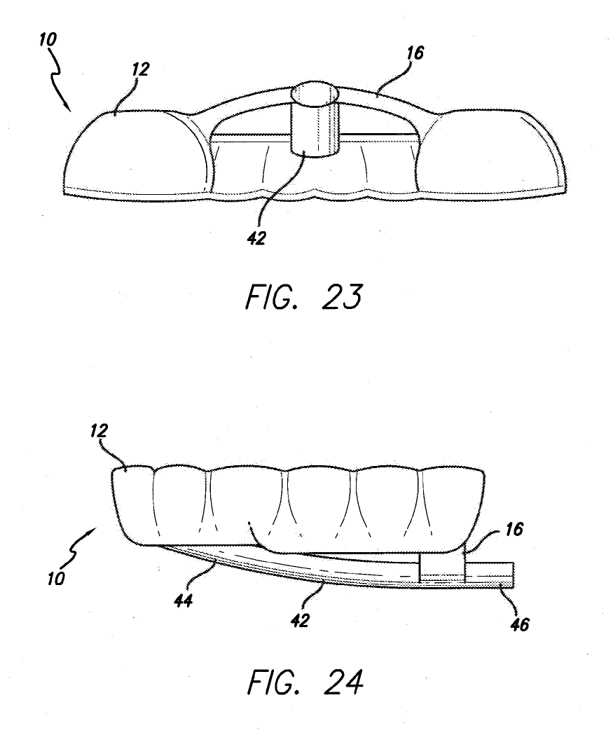

Referring now to FIGS. 22-24, there is shown an embodiment which is the same as FIG. 19 but the whale's tail is not utilized.

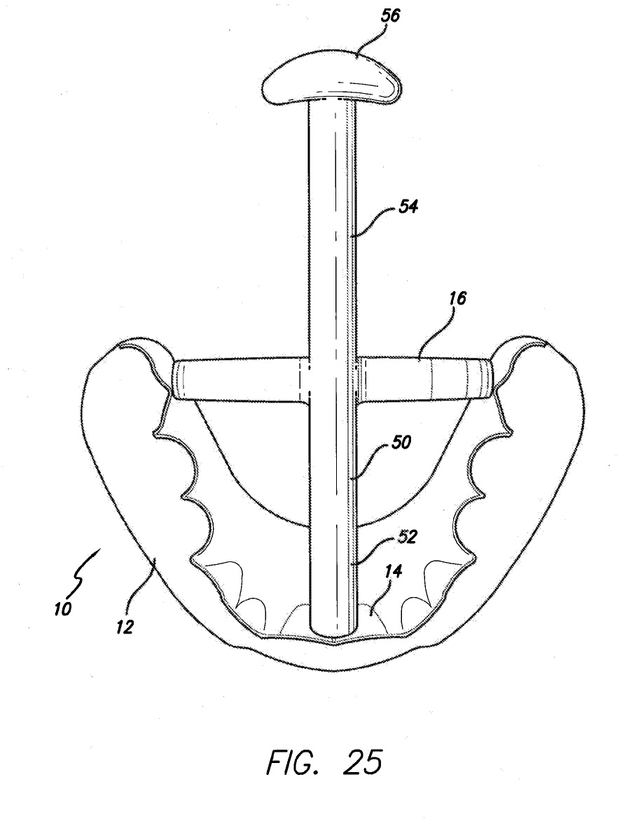

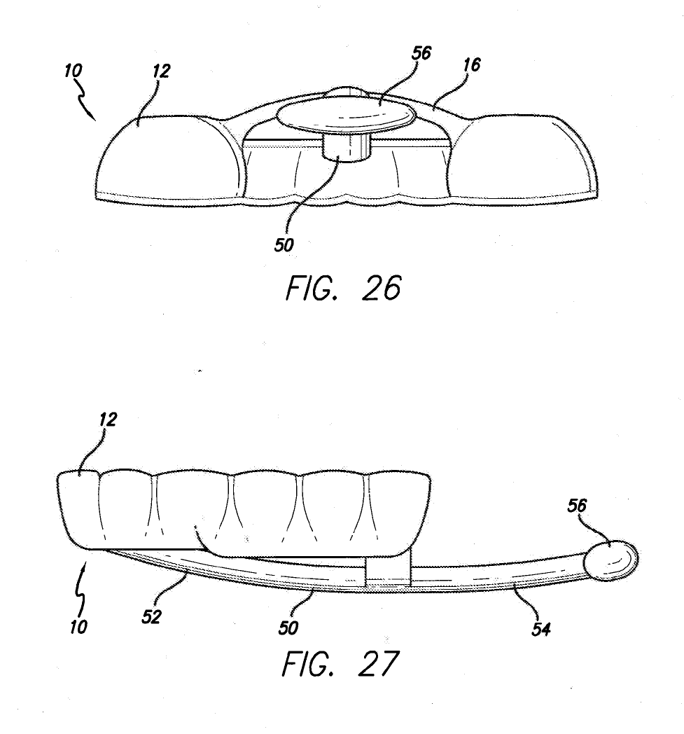

Referring now to FIGS. 25-27, there is shown an embodiment which is similar to FIG. 22, but posterior portion 54 of longitudinal tongue restrainer 50 is elongated. Longitudinal tongue restrainer 50 is formed as part of transpalatal bar 16, as described in FIG. 19. Whale's tail 56 is affixed to the end of posterior portion 54.

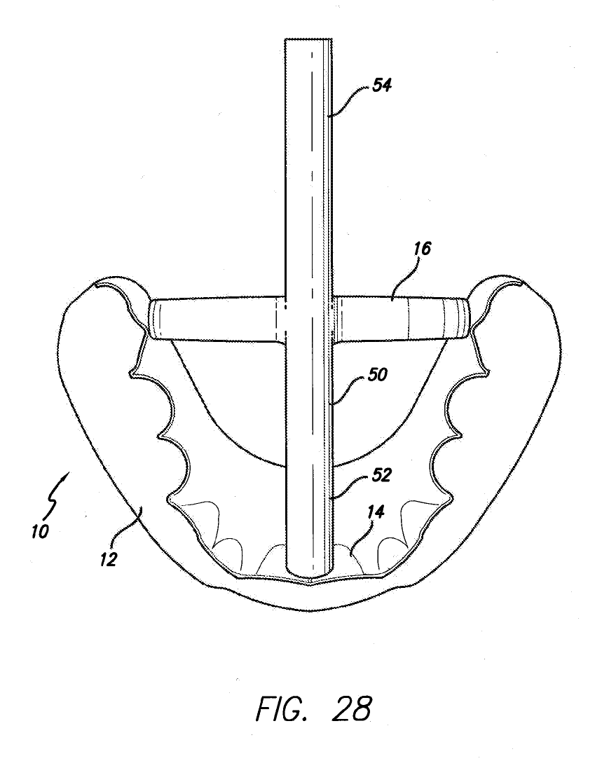

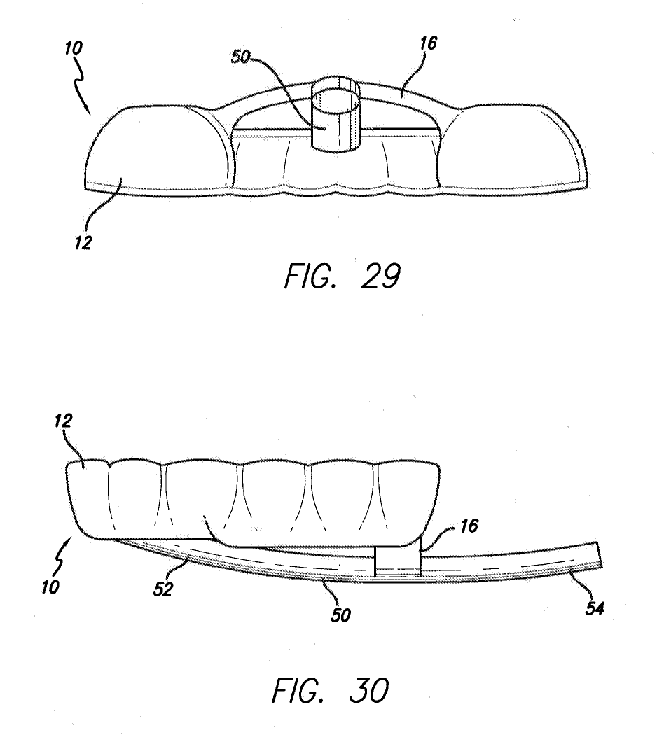

Referring now to FIGS. 28-30, there is shown an embodiment which is the same as FIG. 25 but without the whale's tail.

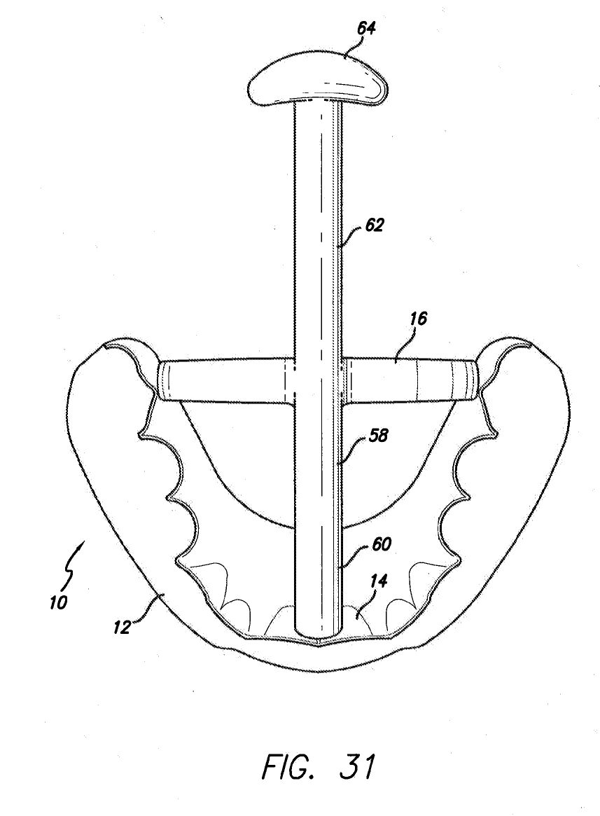

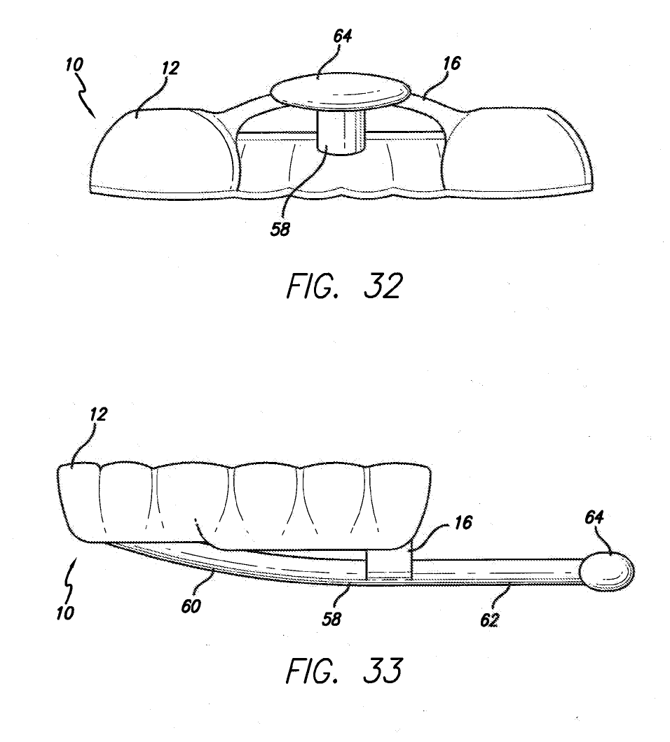

Referring now to FIGS. 31-33, there is shown an embodiment which is similar to FIG. 25, but in which posterior portion 62 of longitudinal tongue restrainer 58 is straight as compared to posterior section 54, which is curved.

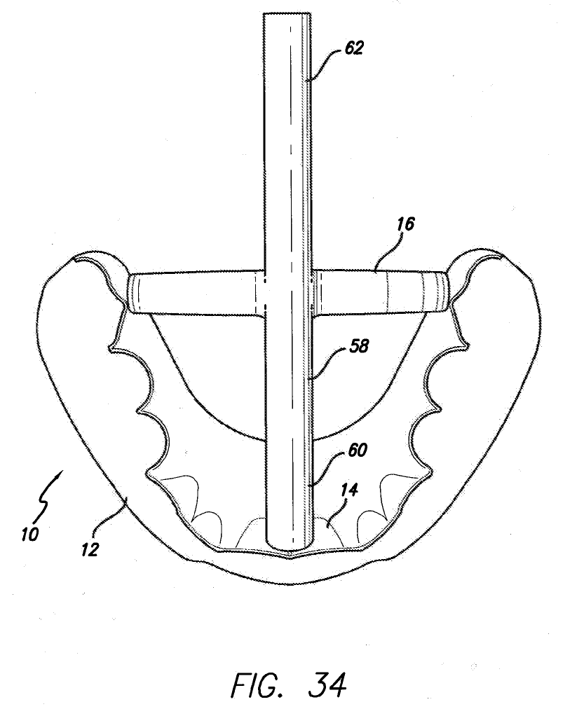

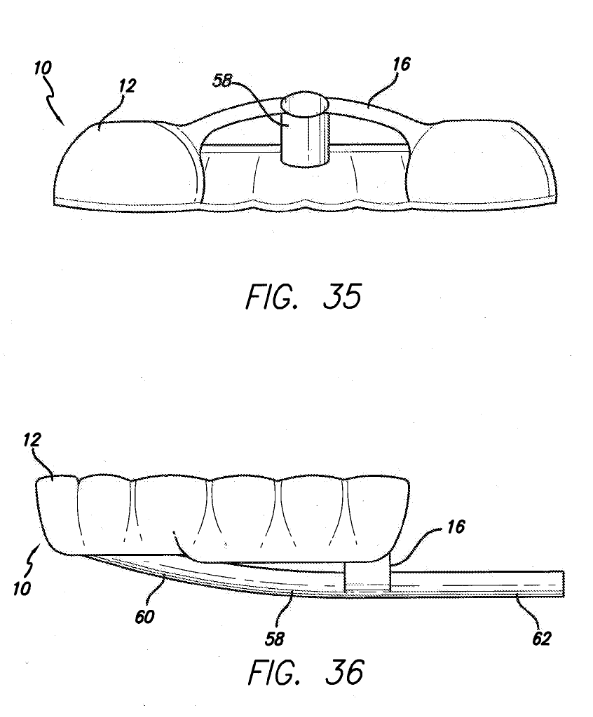

Referring now to FIGS. 34-36, there is shown an embodiment which is the same as FIG. 31, but without the whale's tail.

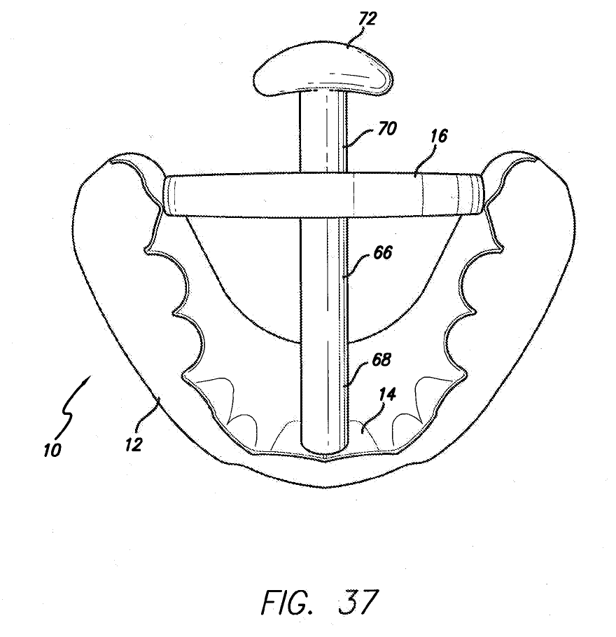

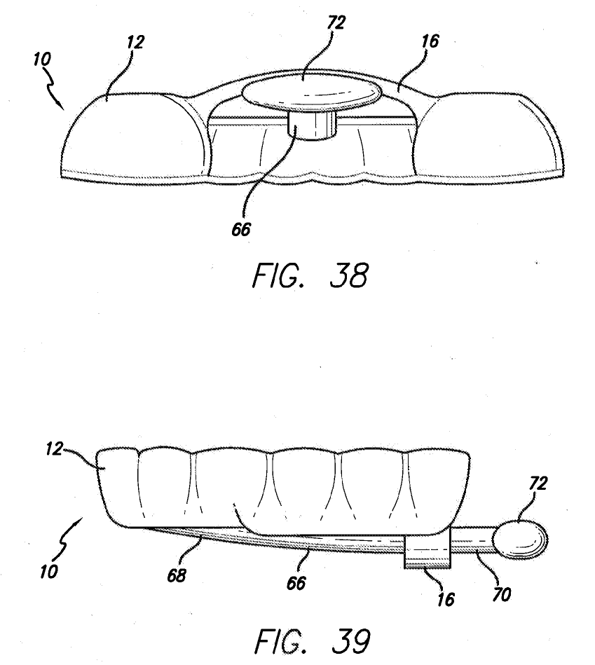

Referring now to FIGS. 37-39, there is shown an embodiment of appliance 10, with body 12, raised anterior strip 14 and transpalatal bar 16. Anterior portion 68 of longitudinal tongue restrainer 66 is attached to anterior strip 14, Posterior portion 70 of longitudinal tongue restrainer 66, rather than passing over or passing through transpalatal bar 16, passes under transpalatal bar 16. Whale's tail 72 is affixed to the end of posterior portion 70.

As can be seen from the embodiments shown, the longitudinal tongue restrainer may pass over, under or through the transpalatal bar. When it passes over the transpalatal bar, it lies closer to the palate, when it passes under the transpalatal bar it lies closer to the tongue, and when it passes through the transpalatal bar it lies in the middle. In each of these positions, the longitudinal tongue restrainer does not touch the palate and there is a gap between the longitudinal tongue restrainer and the palate. In each of these positions, the longitudinal tongue restrainer may or may not touch the tongue. When required for clinical success, the longitudinal tongue restrainer may press down on the tongue to aid in opening the oropharyngeal airway.

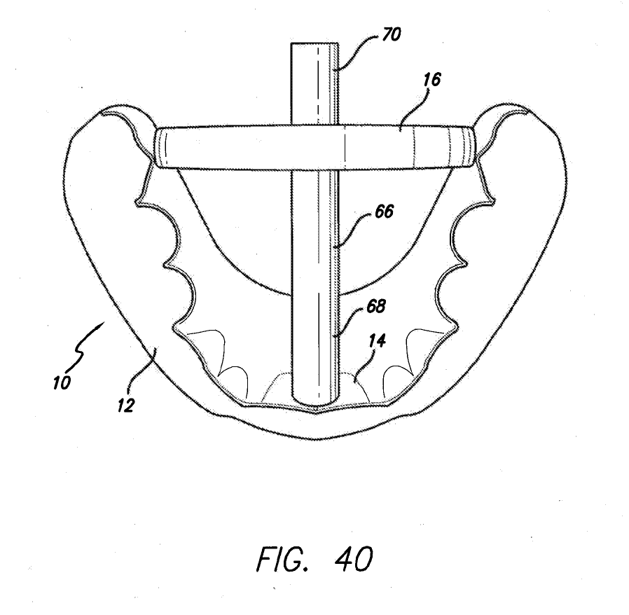

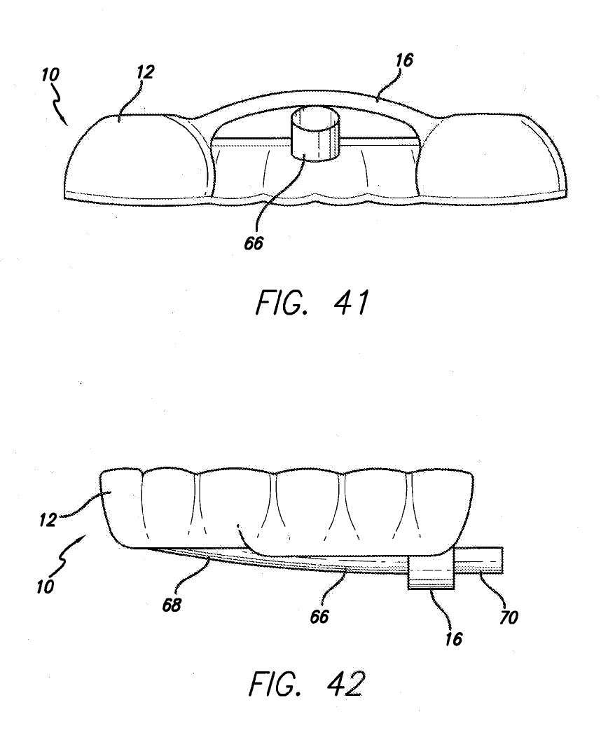

Referring now to FIGS. 40-42, there is shown an embodiment which is the same as FIG. 37 but without the whale's tail.

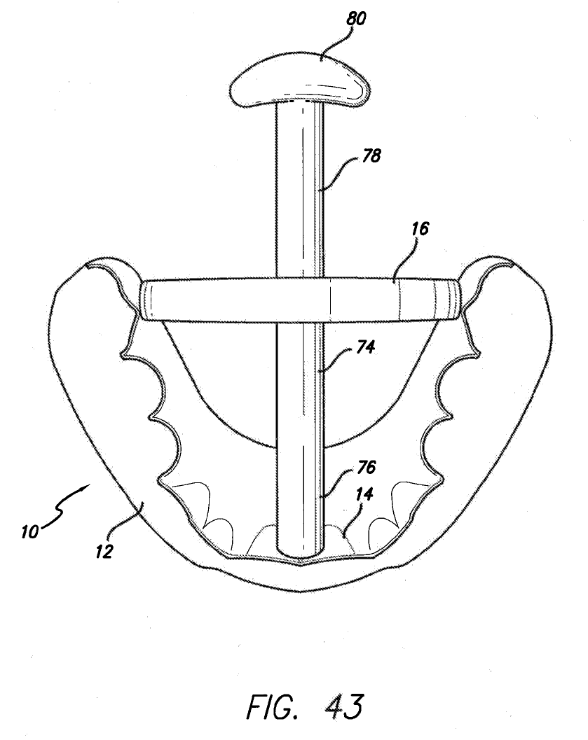

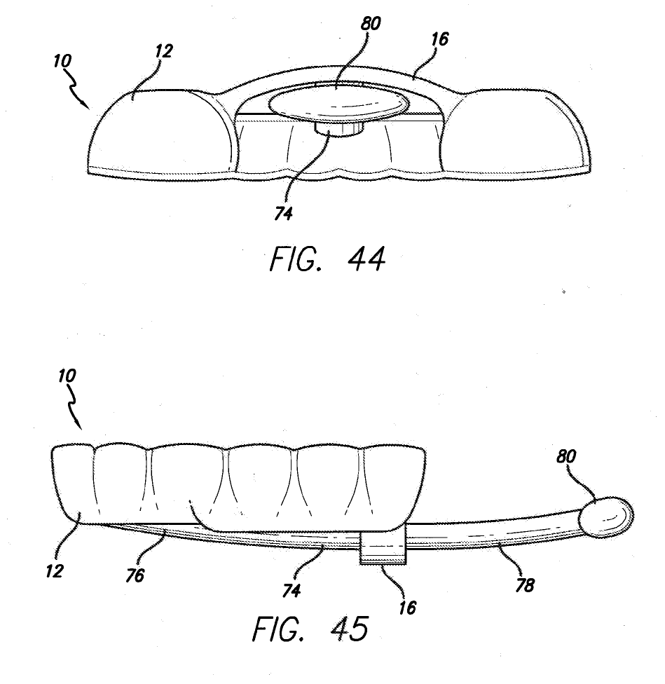

Referring now to FIGS. 43-45, there is shown an embodiment which is similar to FIG. 37 but posterior portion 78 of longitudinal tongue restrainer 74 is elongated.

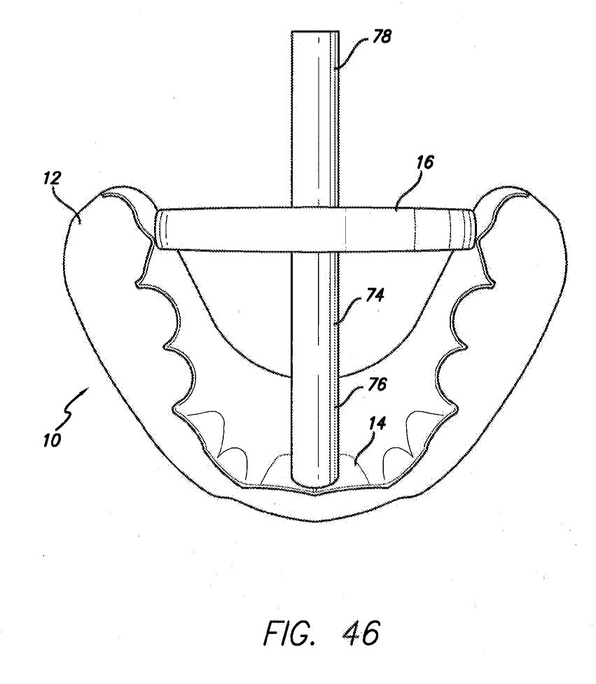

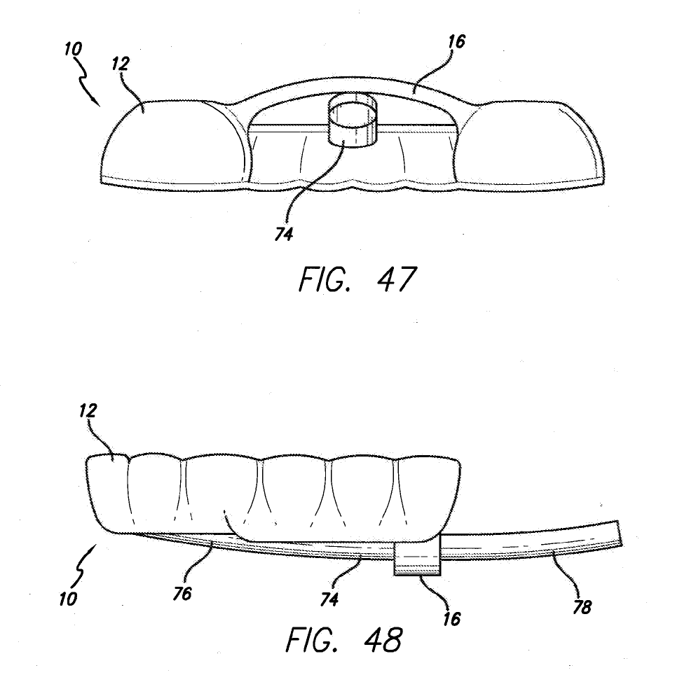

Referring now to FIGS. 46-48, there is shown an embodiment which is the same as FIG. 43 but without the whale's tail.

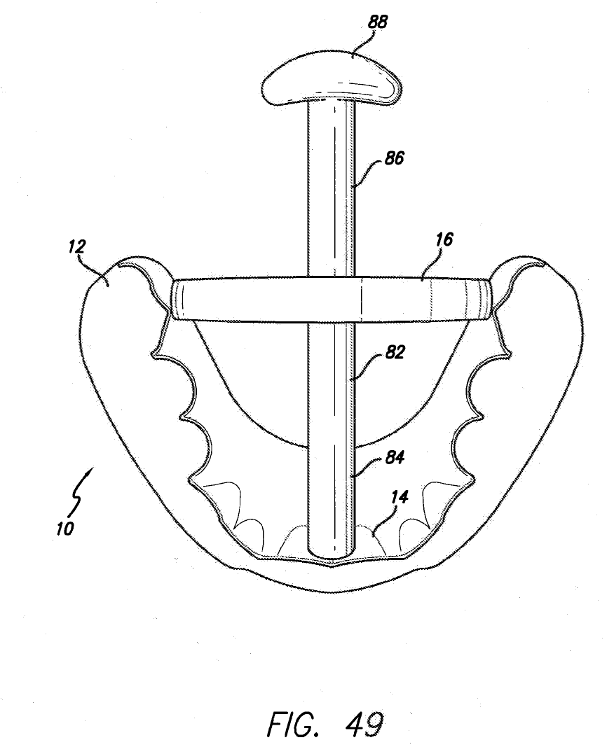

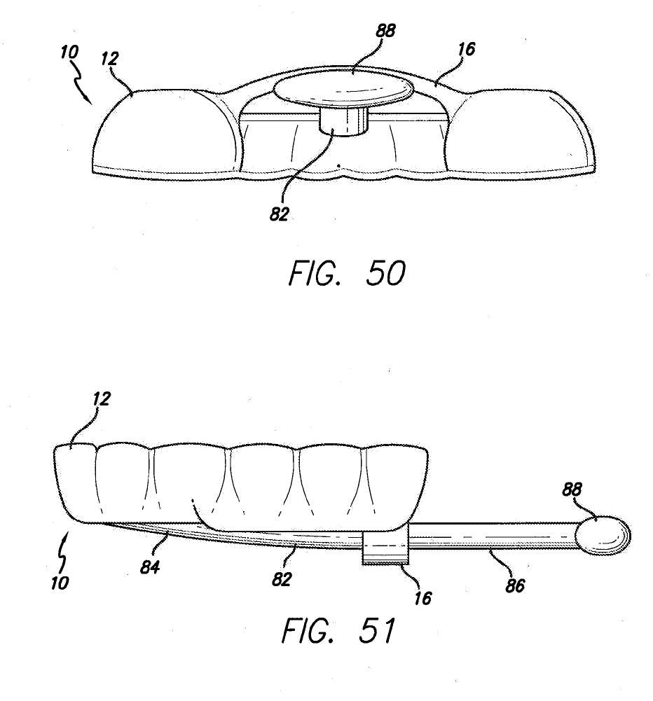

Referring now to FIGS. 49-51, there is shown an embodiment which is similar to FIG. 43, but posterior portion 86 of longitudinal tongue restrainer 82 is straight rather than curved as is posterior portion 78.

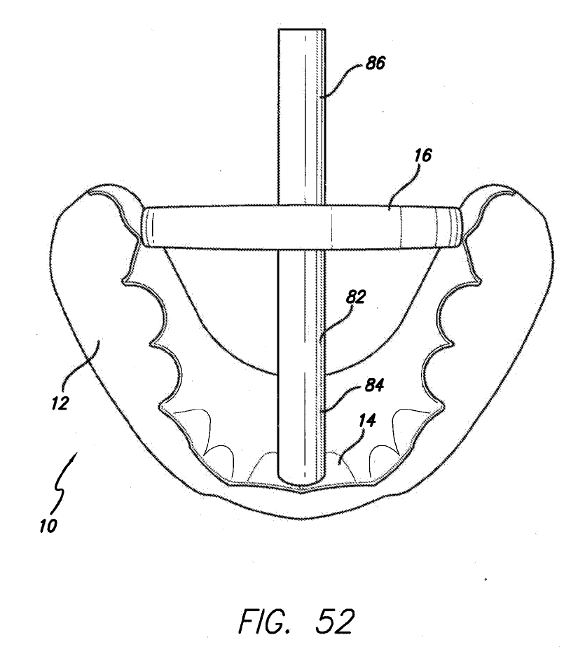

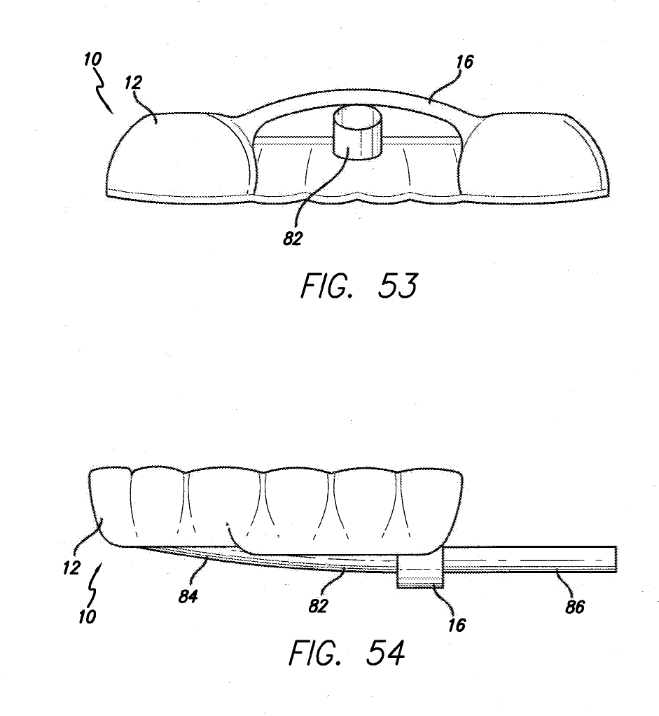

Referring now to FIGS. 52-54, there is shown an embodiment which is the same as FIG. 49, but without whale's tail 88.

In all of the embodiments above described, the longitudinal tongue restrainer does not touch the tongue in most cases. There is a gap between the longitudinal tongue restrainer and the palate The longitudinal tongue restrainer does inhibit and restrain the upward and backward movement of the tongue during sleep by blocking the upward and backward movement of the tongue.

However, the longitudinal tongue restrainer can be designed, by adjustment of the placement and curvature, so that a portion presses on the tongue at only one point or the longitudinal tongue restrainer can be designed so that a portion presses down on the tongue for any length from one point to its entire length, depending upon the needs of the patient.

This embodiment is used only for patients who do not have success in reducing snoring using any of the prior described appliances, in which no part of the appliances touch the tongue in its normal position.

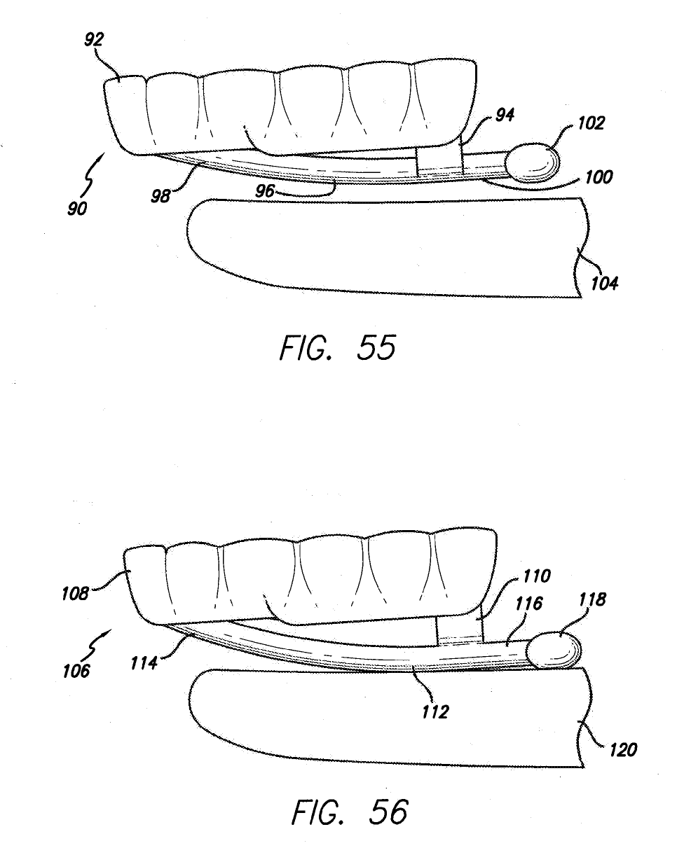

Referring now to FIGS. 55 and 56, there is shown an embodiment which attaches to the upper teeth, rather than the lower teeth. FIG. 55 shows an appliance 90 having a body 92 and a transpalatal bar 94. The appliance fits on the upper teeth like an occlusal coverage. A longitudinal tongue restrainer 96 with anterior portion 98 and posterior portion 100 attaches to the front of body 12 or to a raised anterior discluder strip, as previously described. Longitudinal tongue restrainer 96 has a whale's tale 102 and passes through transpalatal bar 16 and does not touch tongue 104 in its normal state. It will restrain and inhibit the upward and backward movement of the tongue.

Referring now to FIG. 56, there is shown an appliance 106 with body 108 similar to FIG. 55. This appliance is designed for the patients who cannot obtain beneficial results unless the tongue 120 is pressed downward by the appliance. To achieve this result, longitudinal tongue restrainer 112 has anterior portion 114, which attaches to the front of the appliance or a discluder strip, and posterior portion 116 which passes under transpalatal bar 16 curving downward to press against tongue 120. Longitudinal tongue restrainer 112 may be designed to just hold tongue 120 down or it can press tongue 120 down some distance, depending upon the needs of the patient. Longitudinal tongue restrainer 112 can be designed, by placement and curvature, so that only a small portion presses on the tongue at just one point or it can be designed so that a portion presses down on the tongue for any length, from one point to its entire length, depending upon the needs of the patient.

This embodiment is used only for a group of patients who do not have success in reducing snoring using any of the prior described appliances, in which no part of the appliances touch the tongue in its normal position.

As described above with respect to the appliances that fit on the lower teeth, the upper teeth appliances described can be fitted with longitudinal tongue restrainers which curve or are straight, have whale's tails or do not, and pass above, below or through the transpalatal bar, all depending upon the needs of the patient. In addition, the longitudinal tongue restrainer can be flat in shape, as opposed to tubular in shape, as shown. Also, the whale's tail can be curved as shown or can be tubular or have straight edges, depending upon the choice of the medical practitioner and the needs of the patient.

On any of the above-described embodiments, it may be necessary to advance the mandible to increase the airway even more and then acrylic is added to the most lingual portion of the anterior ramp 14, creating a projection wall that comes off of the anterior ramp at 90 degrees. The lower anterior teeth swing forward and bite forward of this lingual wall. This results in the mandible coming forward to obtain an increased opening.

Any of the embodiments described herein may use posterior ramps in place of an anterior strip to disclude the upper and lower teeth. In this device, a base plate is placed over the right and left posterior ramps, anywhere from the 2^{nd} molar to the 1^{st} bicuspid and locked in place with acrylic. In this instance the lower teeth are advanced forward so that the upper and lower incisors are even, edge-to-edge.

All of the devices described herein, which are totally or partially made of plastic, are preferably made of acrylic plastic or thermal plastic or a combination thereof.

### INDUSTRIAL APPLICABILITY

The intra-oral device of this invention may be sold to any person who suffers from a snoring problem or a sleep apnea problem, to effectively reduce or eliminate snoring or sleep apnea.

Having thus described the invention, I Claim:

1-32. (canceled)

33. A dental oral appliance to open the airway for a sleeping individual who suffers with at least one of snoring and obstructive sleep apnea, comprising, a body, structure configured to removably affix the appliance to the upper or lower teeth, structure configured to prevent occlusion of the upper and lower teeth, a transpalatal member configured to provide a gap between the transpalatal member and the palate, and a gap between the transpalatal member and the tongue, extending from the inside of one or more of the right molars to the inside of one or more of the left molars, and a longitudinal tongue restrainer extending from the front of the body portion rearwardly, past the transpalatal member, said longitudinal tongue restrainer attached at one end to the front of the body portion or to the structure to prevent occlusion of the upper and lower teeth, and also attached to the transpalatal member, the transpalatal member and the longitudinal tongue restrainer adapted to restrain the tongue from upward and backward movement

34. The dental oral appliance of claim 33 in which the structure to removably affix the appliance to the upper or lower teeth comprises an occlusal coverage.

35. The dental oral appliance of claim 33 in which the structure to prevent occlusion of the upper and lower teeth comprises a raised incisor ramp that extends from two or more incisors to the lingual.

36. The dental oral appliance of claim 33 in which the structure to prevent occlusion of the upper and lower teeth comprises raised posterior ramps.

37. The dental oral appliance of claim 33 in which the transpalatal member is curved.

38. The dental oral appliance of claim 33 in which the longitudinal tongue restrainer is curved.

39. The dental oral appliance of claim 33 further comprising a perpendicular cross bar attached to the rearward end of the longitudinal tongue restrainer.

40. The dental oral appliance of claim 33 in which the entire appliance is made of an acrylic plastic, thermal plastic or a combination thereof.

41. The dental oral appliance of claim 33 in which the longitudinal tongue restrainer is attached to the tap, bottom or through the middle of the transpalatal member.

42. The dental oral appliance of claim 33 further comprising structure to advance the mandible.

43. The dental oral appliance of claim 33 in which the longitudinal tongue restrainer is cylindrical in shape.

44. The dental oral appliance of claim 33 in which neither the longitudinal tongue restrainer nor the transpalatal member touches the tongue in its normal state but both inhibit the upward and backward movement of the tongue during sleep.

45. The dental oral appliance of claim 33 in which the longitudinal tongue restrainer presses down on the tongue.

46. The dental oral appliance of claim 45 in which the longitudinal tongue restrainer presses down on the tongue a distance of from one point to its entire length.

47. The dental oral appliance of claim 33 in which the longitudinal tongue restrainer is curved downwardly to press down on the tongue, to further open the airway.
